(19) United States
(12) Patent Application Publication
(10) Pub. No.: US 2013/0197355 A1
(43) Pub. Date: Aug. 1, 2013

(54) METHOD OF CONTROLLING NEEDLE GUIDE APPARATUS, AND ULTRASOUND DIAGNOSTIC APPARATUS USING THE SAME

(71) Applicants: Jun-kyo LEE, Gangwon-do (KR); Sung-yoon Kim, Gangwon-do (KR)

(72) Inventors: Jun-kyo LEE, Gangwon-do (KR); Sung-yoon Kim, Gangwon-do (KR)

(73) Assignee: SAMSUNG MEDISON CO., LTD.

(21) Appl. No.: 13/543,328

(22) Filed: Jul. 6, 2012

(30) Foreign Application Priority Data
Feb. 1, 2012 (KR) 10-2012-0010379

(51) Int. Cl. A61B 10/02; A61B 8/13

(52) U.S. Cl. 600/424

(57) ABSTRACT

A method of controlling a needle guide apparatus for assisting insertion of a needle, the method including: setting target coordinates in which the tip of the needle is located inside an object to be diagnosed; and adjusting a position of the needle guide apparatus based on the set target coordinates, wherein the needle guide apparatus is connected to a probe for capturing an ultrasound image of the object to be diagnosed.

### CROSS-REFERENCE TO RELATED PATENT APPLICATION

This application claims the benefit of Korean Patent Application No. 10-2012-0010379, filed on Feb. 1, 2012, in the Korean Intellectual Property Office, the disclosure of which is incorporated herein in its entirety by reference.

### BACKGROUND OF THE INVENTION

1. Field of the Invention

The present invention relates to a method of controlling a needle guide apparatus for assisting a user to insert a needle, and an ultrasound diagnostic apparatus using the same.

2. Description of the Related Art

Ultrasound diagnostic apparatuses transmit an ultrasound signal from the surface of a human body to be diagnosed towards a predetermined part in the inside of the human body and capture a tomogram of a soft tissue or an image of a hematocele by using information of the ultrasound signal reflected from the predetermined portion.

Such ultrasound diagnostic apparatuses have advantages of miniaturized, cheap, and real-time displayable. In addition, ultrasound diagnostic apparatuses has an advantage of high stability due to non-radiation of X rays, and accordingly, are widely used together with X-ray diagnostic apparatuses, Computerized Tomography (CT) scanners, Magnetic Resonance Imaging (MRI) apparatuses, and nuclear medicine diagnostic apparatuses.

In general, an ultrasound biopsy has been popularly performed for tumor diagnosis. A biopsy indicates that a small portion of a tissue in an affected part of a patient is cut off and observed with a naked eye or a microscope. When a biopsy is performed, a malpractice may occur because an operator cannot perceive a trajectory of a needle and a correct position of a needle tip.

To prevent a malpractice in a biopsy, techniques of viewing the tip and a trajectory of an ultrasound needle using a magnetic field sensor have been developed. However, since the magnetic field sensor is located at the other end of the needle instead of a part inserted into a human body, when the needle is bent or damaged inside the human body, an ultrasound diagnostic apparatus may display a wrong position of the needle to a user.

Thus, an apparatus for guiding a user to correctly insert a needle on a position desired by the user and guiding even a user unfamiliar with a biopsy to quickly and correctly insert a needle is required.

### SUMMARY OF THE INVENTION

According to an aspect of the present invention, there is provided a method of controlling a needle guide apparatus for assisting insertion of a needle, the method including: setting target coordinates in which the tip of the needle is located inside an object to be diagnosed; and adjusting a position of the needle guide apparatus based on the set target coordinates, wherein the needle guide apparatus is connected to a probe for capturing an ultrasound image of the object to be diagnosed.

The method may further include receiving the target coordinates in the ultrasound image of the object to be diagnosed from a user.

The method may further include: acquiring a position of the probe; and adjusting the position of the needle guide apparatus based on the acquired position of the probe and the target coordinates.

The method may further include rotating the needle guide apparatus by a predetermined angle based on the set target coordinates.

The method may further include providing a length of the needle required from the needle guide apparatus to the target coordinates.

The method may further include displaying a predicted path along which the needle is inserted up to the target coordinates

The method may further include displaying a position of the needle inserted into the object to be diagnosed.

According to another aspect of the present invention, there is provided an ultrasound diagnostic apparatus including: a needle guide apparatus for assisting insertion of a needle; a coordinate setup unit for setting target coordinates in which the tip of the needle is located inside an object to be diagnosed; and a controller for adjusting a position of the needle guide apparatus based on the set target coordinates, wherein the needle guide apparatus is connected to a probe for capturing an ultrasound image of the object to be diagnosed.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of an ultrasound diagnostic apparatus according to an embodiment of the present invention;

FIG. 2 is a block diagram of an ultrasound diagnostic apparatus according to another embodiment of the present invention;

FIG. 3 is a flowchart illustrating a method of controlling a needle guide apparatus, according to an embodiment of the present invention;

FIG. 4 is a flowchart illustrating a method of controlling the needle guide apparatus based on a position of a probe, according to an embodiment of the present invention;

FIG. 5 is a conceptual diagram of the needle guide apparatus according to an embodiment of the present invention; and

FIGS. 6A to 6C are conceptual diagrams of the needle guide apparatus of which an angle is changed according to a position of a probe, according to an embodiment of the present invention.

### DETAILED DESCRIPTION OF THE INVENTION

Although terms used in the present invention are selected with general terms popularly used at present under the consideration of functions in the present invention, the terms may vary according to the intention of those of ordinary skill in the art, judicial precedents, or introduction of new technology. In addition, in a specific case, the applicant voluntarily may select terms, and in this case, the meaning of the terms is disclosed in a corresponding description part of the invention. Thus, the terms used in the present invention should be defined not by the simple names of the terms but by the meaning of the terms and the contents throughout the present invention.

In the specification, when a certain part ‘includes’ a certain component, this means that the part may further include other components instead of excluding another component unless the context clearly dictates otherwise. In addition, the term, such as ‘ . . . unit’ or ‘module’, disclosed in the specification means a unit for processing at least one function or operation, which may be implemented by hardware, software, or a combination of hardware and software.

In the specification, an ‘ultrasound image’ indicates an image of an object to be diagnosed, which is acquired using an ultrasound wave. The ‘object to be diagnosed’ may mean a portion of a human body. For example, the object to be diagnosed may include an organ, such as a liver, a heart, or a womb, or an unborn child.

In the specification, a ‘user’ may be a doctor, a nurse, a clinical pathologist, or a medical image expert as a medical expert but is not limited thereto.

The present invention will now be described more fully with reference to the accompanying drawings, in which exemplary embodiments of the invention are shown. The invention may, however, be embodied in many different forms and should not be construed as being limited to the embodiments set forth herein. In the drawings, parts not concerned with the description of the present invention are omitted to clearly describe the present invention, and like reference numerals are used to refer to like elements throughout the specification.

As used herein, the term “and/or” includes any and all combinations of one or more of the associated listed items. Expressions such as “at least one of,” when preceding a list of elements, modify the entire list of elements and do not modify the individual elements of the list.

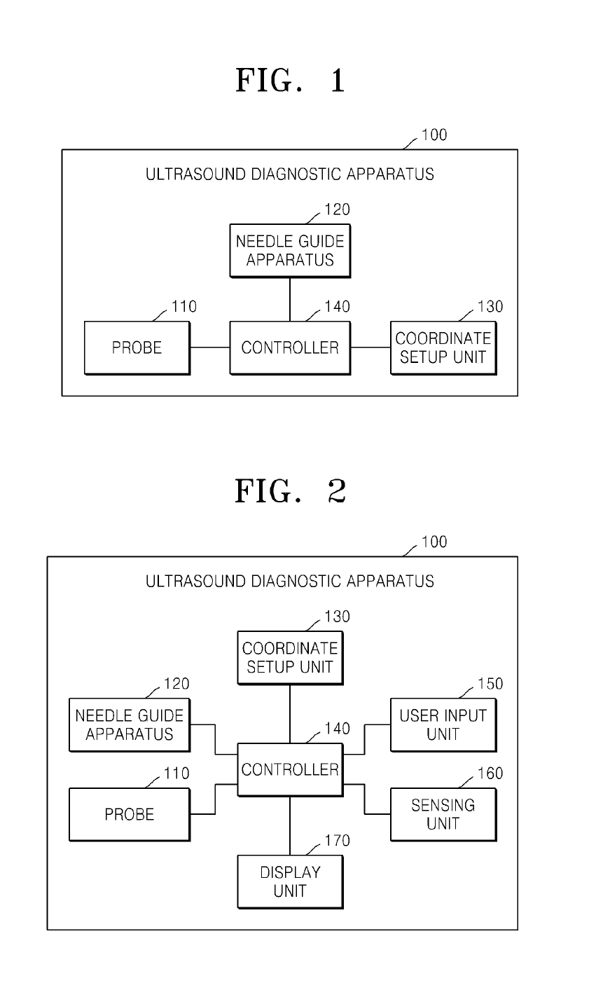

FIG. 1 is a block diagram of an ultrasound diagnostic apparatus 100 according to an embodiment of the present invention.

Referring to FIG. 1, the ultrasound diagnostic apparatus 100 means a device capable of acquiring an ultrasound image from an object to be diagnosed by using an ultrasound wave and guiding a user to perform a biopsy.

The ultrasound diagnostic apparatus 100 can be implemented in various forms. For example, the ultrasound diagnostic apparatus 100 described in the specification may be implemented in a form of a fixed terminal or a mobile terminal. Examples of the mobile terminal are a laptop computer, a Personal Digital Assistant (PDA), and a tablet computer.

According to an embodiment of the present invention, the ultrasound diagnostic apparatus 100 may include a probe 110, a needle guide apparatus 120, a coordinate setup unit 130, and a controller 140. However, not all of the components shown in FIG. 1 are mandatory components. That is, the ultrasound diagnostic apparatus 100 may be implemented by more or less components than the components shown in FIG. 1.

The probe 110 may transmit au ultrasound signal to the object to be diagnosed. The probe 110 may also receive an ultrasound echo signal from the object to be diagnosed. The probe 110 may be at least one of a one-dimensional (1D) probe, a 2D probe, a 3D probe, and a 4D probe.

The ultrasound diagnostic apparatus 100 may generate an ultrasound image of the object to be diagnosed based on the received ultrasound echo signal. The ultrasound image may include all dimensional images, such as a 1D image, a 2D image, a 3D image, and a 4D image.

The probe 110 may transmit an ultrasound signal to a needle inserted into the object to be diagnosed and receive an ultrasound echo signal reflected from the needle, thereby tracking a position of the inserted needle.

The needle guide apparatus 120 is a device for assisting insertion of the needle so that a user can insert the needle on a correct position. The needle guide apparatus 120 may include a needle insertion hole. The needle guide apparatus 120 may be connected to one side of the probe 110 or may exist at a position penetrating the probe 110.

A position of the needle guide apparatus 120 may be automatically adjusted by the controller 140 or manually adjusted by the user. In addition, the position of the needle guide apparatus 120 may be adjusted in various directions, such as to the top and the bottom, to the left and right, or to the front and rear.

The coordinate setup unit 130 may set target coordinates in which the tip of the needle is located inside the object to be diagnosed. According to an embodiment of the present invention, the coordinate setup unit 130 may set target coordinates based on a coordinate value input by the user.

The controller 140 may adjust a position of the needle guide apparatus 120 based on the set target coordinates. According to an embodiment of the present invention, the controller 140 may adjust a position of the needle guide apparatus 120 by further considering a position of the probe 110. That is, the controller 140 may adjust a position of the needle guide apparatus 120 based on the position of the probe 110 and the set target coordinates.

The position of the needle guide apparatus 120 may include an angle formed by the needle guide apparatus 120 and the probe 110 and a direction of the needle guide apparatus 120 oriented to the object to be diagnosed.

According to an embodiment of the present invention, the controller 140 may rotate the needle guide apparatus 120 by a predetermined angle so that the needle is correctly inserted on the target coordinates inside the object to be diagnosed.

The controller 140 may calculate a length of the needle required from the needle guide apparatus 120 to the target coordinates inside the object to be diagnosed. In addition, the controller 140 may calculate a position of the needle inserted into the object to be diagnosed by using an ultrasound echo signal reflected from the needle.

The controller 140 may generally control the probe 110, the needle guide apparatus 120, and the coordinate setup unit 130.

FIG. 2 is a block diagram of the ultrasound diagnostic apparatus 100 according to another embodiment of the present invention.

As shown in FIG. 2, the ultrasound diagnostic apparatus 100 according to another embodiment of the present invention may further include a user input unit 150, a sensing unit 160, and a display unit 170 besides the probe 110, the needle guide apparatus 120, the coordinate setup unit 130, and the controller 140. The configuration shown in FIG. 1 is not described herein.

The user input unit 150 generates input data for a user to control an operation of the ultrasound diagnostic apparatus 100. The user input unit 150 may include a keypad, a dome switch, a touch pad (pressure/electrostatic), a jog wheel, a jog switch, etc. In particular, when the touch pad forms a layer structure with the display unit 170, this may be called a touch screen.

The user input unit 150 may receive target coordinates in which the tip of the needle is located inside the object to be diagnosed from the user. According to an embodiment of the present invention, the user may select target coordinates in a displayed ultrasound image of the object to be diagnosed with use of the user input unit 150.

The sensing unit 160 may generate a sensing signal for controlling an operation of the ultrasound diagnostic apparatus 100 by sensing a current state of the ultrasound diagnostic apparatus 100. For example, the sensing unit 160 may acquire a position of the probe 110. When the probe 110 is moving or the position of the probe 110 is changed, the sensing unit 160 may immediately detect it.

The display unit 170 displays information processed by the ultrasound diagnostic apparatus 100. For example, the display unit 170 may provide a length of the needle required from the needle guide apparatus 120 to target coordinates in which the tip of the needle is located inside the object to be diagnosed. The display unit 170 may display a predicted insertion path of the needle from the needle guide apparatus 120 to the target coordinates.

The display unit 170 may display an actual position of the needle inserted into the object to be diagnosed. According to an embodiment of the present invention, the display unit 170 may display a trajectory and/or a current position of the needle inserted into the object to be diagnosed.

As described above, when the display unit 170 and the touch pad form the layer structure to be a touch screen, the display unit 170 may be used as an input device in addition to an output device. The display unit 170 may include at least one of a Liquid Crystal Display (LCD), a Thin Film Transistor-Liquid Crystal Display (TFT-LCD), an Organic Light Emitting Diode (OLED), a flexible display, and a 3D display. Two or more display units 170 may exist according to an implementation form of the ultrasound diagnostic apparatus 100.

The touch screen may be configured to detect a touch input position, a touched area, and a touch input pressure. In addition, the touch screen may be configured to detect a real-touch and a proximity-touch.

In the specification, the real-touch indicates that a pointer actually touches a screen, and the proximity-touch indicates that the pointer accesses the screen with a predetermined distance without actually touching the screen. In the specification, the pointer is a tool for real-touching or proximity-touching a specific part of a displayed screen. Examples of the pointer are a stylus pen and a finger.

Although not shown, to detect the real-touch or the proximity-touch on the touch screen, various sensors may be included inside or near the touch screen. Alternatively, the various sensors for detecting the real-touch or the proximity-touch on the touch screen may be disposed in the sensing unit 160.

The controller 140 may generally control the user input unit 150, the sensing unit 160, and the display unit 170 in addition to the probe 110, the needle guide apparatus 120, and the coordinate setup unit 130.

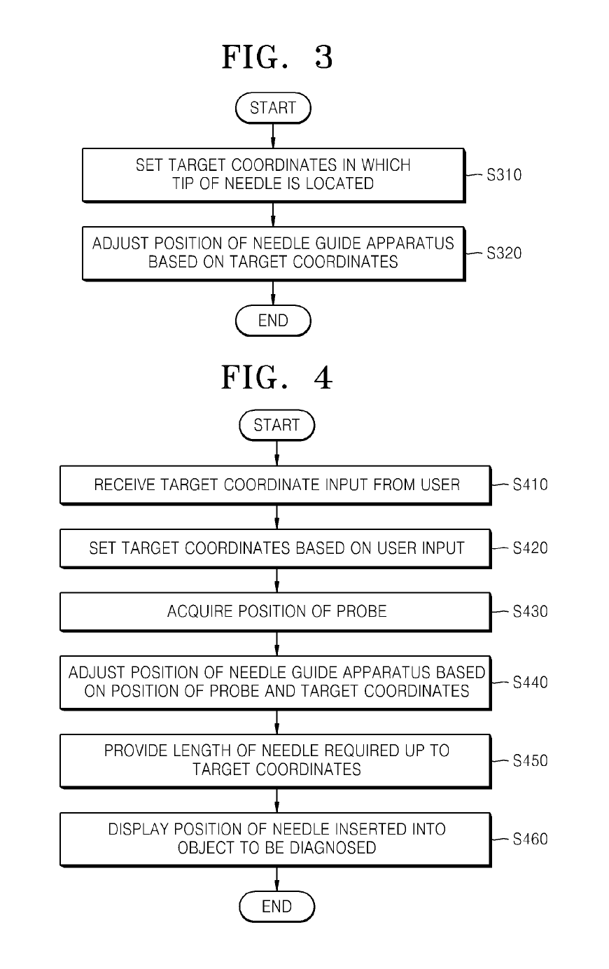

Methods of controlling a position of the needle guide apparatus 120 for a user to correctly insert the needle into an object to be diagnosed by using the configurations of the ultrasound diagnostic apparatus 100 will now be described in detail with reference to FIGS. 3 and 4.

FIG. 3 is a flowchart illustrating a method of controlling the needle guide apparatus 120, according to an embodiment of the present invention.

Referring to FIG. 3, the method includes operations sequentially processed by the ultrasound diagnostic apparatus 100 shown in FIG. 1. Thus, although omitted hereinafter, the description of the ultrasound diagnostic apparatus 100 shown in FIG. 1 is also applied to the method of FIG. 3.

As shown in FIG. 3, in operation S310, the ultrasound diagnostic apparatus 100 sets target coordinates in which the tip of the needle is located inside an object to be diagnosed. The target coordinates may indicate a position of a tissue, such as a tumor, for which a biopsy is needed.

According to an embodiment of the present invention, the ultrasound diagnostic apparatus 100 may set the target coordinates in which the tip of the needle is located based on a user input.

According to another embodiment of the present invention, the ultrasound diagnostic apparatus 100 may extract the position of the tissue for which a biopsy is needed and recommend the extracted position of the tissue to the user as the target coordinates in which the tip of the needle is located.

In operation S320, the ultrasound diagnostic apparatus 100 adjust a position of the needle guide apparatus 120 based on the set target coordinates. That is, the ultrasound diagnostic apparatus 100 adjusts the position of the needle guide apparatus 120 so that the needle moves towards the target coordinates when the needle is inserted into the needle guide apparatus 120.

For example, the ultrasound diagnostic apparatus 100 may measure a current angle of the needle guide apparatus 120. When the user inserts the needle into the object to be diagnosed with the measured current angle, the tip of the needle may not arrive at the target coordinates. In this case, the ultrasound diagnostic apparatus 100 adjusts an angle and direction of the needle guide apparatus 120 so that the needle can arrive at the target coordinates.

According to an embodiment of the present invention, the user does not have to voluntarily adjust the needle guide apparatus 120 for the needle to be inserted on the target coordinates inside the object to be diagnosed. That is, since the ultrasound diagnostic apparatus 100 can automatically adjust an angle and direction of the needle guide apparatus 120, even a user unfamiliar with a biopsy may correctly acquire a desired tissue.

FIG. 4 is a flowchart illustrating a method of controlling the needle guide apparatus 120 based on a position of the probe 110, according to an embodiment of the present invention.

Referring to FIG. 4, the method includes operations sequentially processed by the ultrasound diagnostic apparatus 100 shown in FIG. 2. Thus, although omitted hereinafter, the description of the ultrasound diagnostic apparatus 100 shown in FIG. 2 is also applied to the method of FIG. 4.

As shown in FIG. 4, in operation S410, the ultrasound diagnostic apparatus 100 receives a target coordinate input from a user. In operation S420, the ultrasound diagnostic apparatus 100 sets or confirms target coordinates based on the user input.

According to an embodiment of the present invention, the ultrasound diagnostic apparatus 100 may display an ultrasound image of an object to be diagnosed to a user. In this case, the user may select target coordinates in the displayed ultrasound image.

An ultrasound image according to an embodiment of the present invention may be at least one of a brightness mode (B mode) image in which an amplitude of an ultrasound echo signal reflected from an object to be diagnosed is represented as brightness, a Doppler mode image in which an image of a moving object to be diagnosed is represented in a spectrum format using a Doppler effect, a motion mode (M mode) image in which a motion of an object to be diagnosed along time at a predetermined position is represented, an elastic mode image in which a reaction difference between when a compression is added to an object to be diagnosed and when the compression is not added to the object to be diagnosed is represented as an image, and a color mode (C mode) image in which a speed of a moving object to be diagnosed is represented as colors using the Doppler effect.

In operation S430, the ultrasound diagnostic apparatus 100 acquires a position of the probe 110 by using a sensor. The sensor may be attached to or separated from the probe 110. In operation S440, the ultrasound diagnostic apparatus 100 adjusts a position of the needle guide apparatus 120 based on the position of the probe 110 and the target coordinates.

In operation S450, the ultrasound diagnostic apparatus 100 provides a length of the needle required from the needle guide apparatus 120 to the target coordinates inside the object to be diagnosed.

For example, when the length of the needle required from the needle guide apparatus 120 to the target coordinates inside the object to be diagnosed is 5 cm, the ultrasound diagnostic apparatus 100 may adjust the position of the needle guide apparatus 120 and display the required length of the needle (e.g., 5 cm). In this case, the user may not insert the needle up to an unnecessary depth by using the needle guide apparatus 120.

In operation S460, the ultrasound diagnostic apparatus 100 displays a position of the needle inserted into the object to be diagnosed.

For example, the ultrasound diagnostic apparatus 100 may acquire position information of the needle inserted into the object to be diagnosed by using an ultrasound signal. The ultrasound diagnostic apparatus 100 may display a current position and/or an insertion path of the needle based on the acquired position information of the needle. In this case, the current position and/or the insertion path of the needle may be overlaid and displayed on the ultrasound image of the object to be diagnosed.

According to an embodiment of the present invention, the ultrasound diagnostic apparatus 100 may display a predicted insertion path from the needle guide apparatus 120 to the target coordinates.

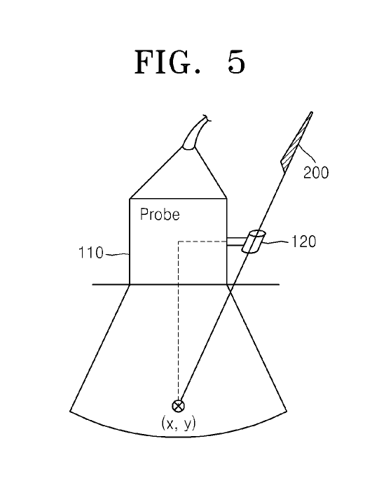

FIG. 5 is a conceptual diagram of the needle guide apparatus 120 according to an embodiment of the present invention.

As shown in FIG. 5, the probe 110 may receive an ultrasound echo signal reflected from an object to be diagnosed. In this case, the ultrasound diagnostic apparatus 100 may display an ultrasound image of the object to be diagnosed based on the ultrasound echo signal.

The needle guide apparatus 120 may be connected to the probe 110. A user may insert a needle 200 into the object to be diagnosed by using the needle guide apparatus 120 to perform a biopsy.

The ultrasound diagnostic apparatus 100 may receive target coordinates of a tissue for which the biopsy is performed from the user. For example, the target coordinates may be (x, y). When the needle 200 is inserted into the object to be diagnosed by means of the needle guide apparatus 120, the ultrasound diagnostic apparatus 100 may adjust a position of the needle guide apparatus 120 so that the needle 200 arrives at the target coordinates (x, y).

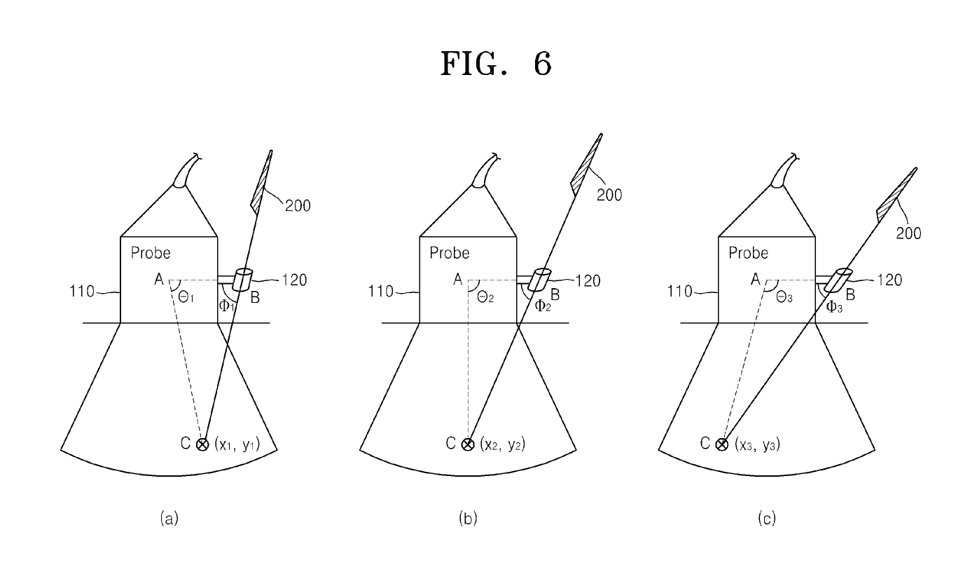

FIGS. 6A to 6C are conceptual diagrams of the needle guide apparatus 120 of which an angle is changed according to a position of the probe 120, according to an embodiment of the present invention.

As shown in FIG. 6A, target coordinates (x1, y1) inside an object to be diagnosed may be located in the relatively right from the center point of the probe 110. Assuming that the center point of the probe 110 is A, the center point of the needle guide apparatus 120 is B, and the target coordinates (x1, y1) are C, an angle formed between a line connecting A and B (hereinafter, ‘segment AB’) and a line connecting A and C (hereinafter, ‘segment AC’) may be θ1.

In this case, the ultrasound diagnostic apparatus 100 may adjust a position of the needle guide apparatus 120 so that an angle formed between a line connecting B and A (hereinafter ‘segment BA’) and a line connecting B and C (hereinafter ‘segment BC’) is φ1.

As shown in FIG. 6B, target coordinates (x2, y2) inside the object to be diagnosed may be located on a direct line from the center point of the probe 110.

Assuming that the center point of the probe 110 is A, the center point of the needle guide apparatus 120 is B, and the target coordinates (x2, y2) are C, an angle formed between the segment AB and the segment AC may be θ2(=90°).

In this case, the ultrasound diagnostic apparatus 100 may adjust a position of the needle guide apparatus 120 so that an angle formed between the segment BA and the segment BC is φ2.

As shown in FIG. 6C, target coordinates (x3, y3) inside the object to be diagnosed may be located in the relatively left from the center point of the probe 110. Assuming that the center point of the probe 110 is A, the center point of the needle guide apparatus 120 is B, and the target coordinates (x3, y3) are C, an angle formed between the segment AB and the segment AC may be θ3.

In this case, the ultrasound diagnostic apparatus 100 may adjust a position of the needle guide apparatus 120 so that an angle formed between the segment BA and the segment BC is φ3.

That is, according to an embodiment of the present invention, when the probe 110 moves in the right based on target coordinates, an angle formed between the segment AB and the segment AC may increase (θ1<θ2<θ3). The ultrasound diagnostic apparatus 100 may adjust a position of the needle guide apparatus 120 so that an angle formed between the segment BA and the segment BC gradually decrease (θ3<θ2<θ1).

The method according to an embodiment of the present invention may be embodied in a program command format executable by various computer means and recorded in a computer-readable recording medium. The computer-readable recording medium may include program commands, data files, data structures, or a combination of them. The program commands recorded in the computer-readable recording medium may be specifically designed and constructed for the present invention or be well known and usable to those of ordinary computer software skill in the art. Examples of the computer-readable recording medium are magnetic media such as hard disks, floppy disks, and magnetic tapes, optical media such as CD-ROMs and DVDs, magneto-optical media such as floptical disks, and hardware devices such as ROMs, RAMs, and flash memories, which are specifically constructed to store and execute program commands. Examples of the program commands are machine language codes created by a compiler and high-level language codes executable by computers using an interpreter.

According to an embodiment of the present invention, the ultrasound diagnostic apparatus 100 may automatically adjust a position of the needle guide apparatus 120 so that a user can correctly insert a needle on target coordinates inside an object to be diagnosed. Thus, even a user unfamiliar with a biopsy can correctly insert a needle on target coordinates.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A method of controlling a needle guide apparatus for assisting insertion of a needle, the method comprising:
setting target coordinates in which the tip of the needle is located inside an object to be diagnosed; and
adjusting a position of the needle guide apparatus based on the set target coordinates,
wherein the needle guide apparatus is connected to a probe for capturing an ultrasound image of the object to be diagnosed.

2. The method of claim 1, wherein the setting of the target coordinates comprises receiving the target coordinates in the ultrasound image of the object to be diagnosed from a user.

3. The method of claim 1, wherein the adjusting of the position of the needle guide apparatus comprises:
acquiring a position of the probe; and
adjusting the position of the needle guide apparatus based on the acquired position of the probe and the target coordinates.

4. The method of claim 1, wherein the adjusting of the position of the needle guide apparatus comprises rotating the needle guide apparatus by a predetermined angle based on the set target coordinates.

5. The method of claim 1, further comprising providing a length of the needle required from the needle guide apparatus to the target coordinates.

6. The method of claim 1, further comprising displaying a predicted path along which the needle is inserted up to the target coordinates.

7. The method of claim 1, further comprising displaying a position of the needle inserted into the object to be diagnosed.

8. An ultrasound diagnostic apparatus comprising:
a needle guide apparatus for assisting insertion of a needle;
a coordinate setup unit for setting target coordinates in which the tip of the needle is located inside an object to be diagnosed; and
a controller for adjusting a position of the needle guide apparatus based on the set target coordinates,
wherein the needle guide apparatus is connected to a probe for capturing an ultrasound image of the object to be diagnosed.

9. The ultrasound diagnostic apparatus of claim 8, further comprising a user input unit for receiving the target coordinates in the ultrasound image of the object to be diagnosed from a user.

10. The ultrasound diagnostic apparatus of claim 8, further comprising a sensing unit for acquiring a position of the probe,
wherein the controller adjusts the position of the needle guide apparatus based on the acquired position of the probe and the target coordinates.

11. The ultrasound diagnostic apparatus of claim 8, wherein the controller rotates the needle guide apparatus by a predetermined angle based on the set target coordinates.

12. The ultrasound diagnostic apparatus of claim 8, further comprising a display unit for providing a length of the needle required from the needle guide apparatus to the target coordinates.

13. The ultrasound diagnostic apparatus of claim 8, further comprising a display unit for displaying a predicted path along which the needle is inserted up to the target coordinates.

14. The ultrasound diagnostic apparatus of claim 8, further comprising a display unit for displaying a position of the needle inserted into the object to be diagnosed.

15. A computer-readable recording medium storing a computer-readable program for executing the method of claim 1.
